(19) United States
(12) Patent Application Publication
(10) Pub. No.: US 2021/0353808 A1
(43) Pub. Date: Nov. 18, 2021

(54) FIELD DISINFECTION MOBILE ROBOT AND CONTROL METHOD THEREOF

(71) Applicants: MICRO-STAR INT'L CO., LTD., New Taipei City (TW); MSI COMPUTER (SHENZHEN) CO., LTD., Shenzhen City (CN)

(72) Inventors: Shih-Che HUNG, Taoyuan City (TW); Chiung-Wen CHANG, Taipei City (TW); Pi-Chang JUNG, New Taipei City (TW); Chien-Ying LI, Keelung City (TW)

(73) Assignees: MICRO-STAR INT'L CO., LTD., New Taipei City (TW); MSI COMPUTER (SHENZHEN) CO., LTD., Shenzhen City (CN)

(21) Appl. No.: 17/005,011

(22) Filed: Aug. 27, 2020

(30) Foreign Application Priority Data
May 14, 2020 (TW) 109115988

(51) Int. Cl. A61L 2/26; G05D 1/00; A61L 2/10

(52) U.S. Cl. CPC A61L 2/26; G05D 1/0016; G05D 1/0094; A61L 2202/25; A61L 2202/11; A61L 2202/16; A61L 2202/14; A61L 2/10

(57) ABSTRACT

A field disinfection mobile robot is provided. The field disinfection mobile robot includes a light-emitting device and a mobile device. The light-emitting device includes at least one lamp. The at least one lamp is configured to emit ultraviolet light. The mobile device carries the light-emitting device and includes at least one wheel and a processing circuit. The processing circuit calculates irradiation time according to a level of a lethal dose and intensity of the ultraviolet light and controls a rotation speed of the at least one wheel according to the irradiation time.

### CROSS REFERENCE TO RELATED APPLICATIONS

This application claims priority of Taiwan Patent Application No. 109115988, filed on May 14, 2020, the entirety of which is incorporated by reference herein.

### BACKGROUND OF THE INVENTION

### Field of the Invention

This invention is related to a field disinfection mobile robot, and in particular it is related to a field disinfection mobile robot which utilizes ultraviolet light to disinfect its environment.

### Description of the Related Art

Ultraviolet light (UV) can destroy the DNA (Deoxyribonucleic acid) of pernicious bacteria and viruses, causing the bacteria and viruses to lose their ability to reproduce and then die. Thus, ultraviolet light is often used as a disinfectant in public places, such as hospitals. Generally, when a user operates ultraviolet light, the user has to place an ultraviolet lamp in a space to be disinfected. After the disinfection is complete, the UV lamp is removed.

### BRIEF SUMMARY OF THE INVENTION

The present invention provides a field disinfection mobile robot. The field disinfection mobile robot comprises a light-emitting device and a mobile device. The light-emitting device comprises at least one lamp. The at least one lamp is configured to emit ultraviolet light. The mobile device carries the light-emitting device and comprises at least one wheel and a processing circuit. The processing circuit calculates irradiation time according to a level of a lethal dose and intensity of the ultraviolet light and controls a rotation speed of the at least one wheel according to the irradiation time.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a schematic diagram showing a field disinfection mobile robot according to an exemplary embodiment of the present invention;

FIG. 2 is a schematic diagram of a map according to an embodiment of the present invention;

FIG. 3A is a schematic diagram of a state of a light-emitting device according to an exemplary embodiment of the present invention;

FIG. 3B is a schematic diagram of another state of a light-emitting device according to an exemplary embodiment of the present invention;

FIG. 4A is a schematic flow chart of a control method according to one exemplary embodiment of the present invention; and

FIG. 4B is a schematic flow chart of a control method according to another exemplary embodiment of the present invention.

### DETAILED DESCRIPTION OF THE INVENTION

The present invention will be described with respect to particular embodiments and with reference to certain drawings, but the invention is not limited thereto and is only limited by the claims. The drawings described are only schematic and are non-limiting. In the drawings, the size of some of the elements may be exaggerated for illustrative purposes and not drawn to scale. The dimensions and the relative dimensions do not correspond to actual dimensions in the practice of the invention.

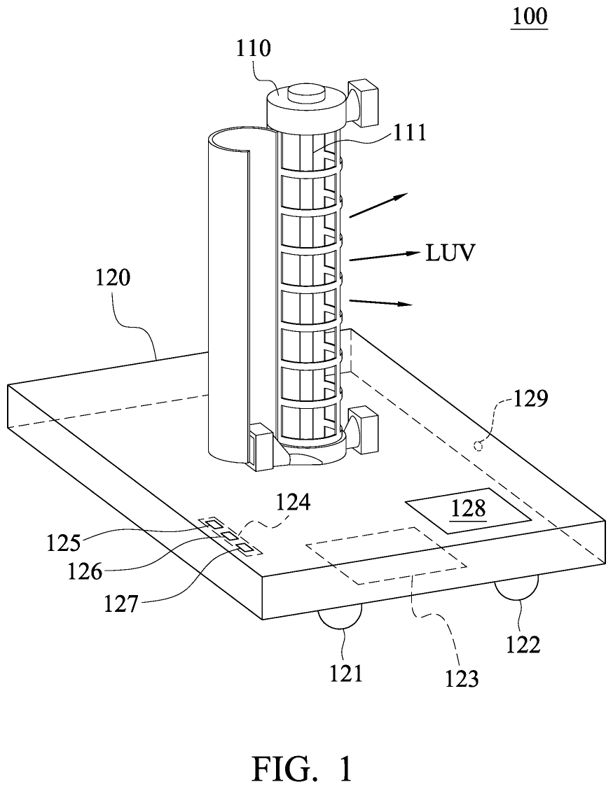

FIG. 1 is a schematic diagram showing a field disinfection mobile robot according to an exemplary embodiment of the present invention. As shown in FIG. 1, a field disinfection mobile robot 100 comprises a light-emitting device 110 and a mobile device 120. The light-emitting device 110 comprises a lamp 111, however, which is not intended to limit the present invention. In other embodiments, the light-emitting device 110 comprises more lamps. In the embodiment, the lamp 110 is used to emit ultraviolet light LUV.

The mobile device 120 carries the light-emitting device 110 and moves while carrying the light-emitting device 110. The ultraviolet light LUV emitted by the light-emitting device 110 provides a sterilization function. Thus, when the mobile device 120 moves while carrying the light-emitting device 110, the ultraviolet light LUV can sterilize the bacteria or viruses in the space through which the robot 100 passes. In the embodiment, the mobile device 120 comprises at least two wheels 121 and 122 and a processing circuit 123.

The processing circuit 123 calculates the irradiation time according to the level of a lethal dose and the intensity of the ultraviolet light LUV. The irradiation time indicates the amount of time that the field disinfection mobile robot 100 stays in a space to be disinfected. For example, in a case where the intensity of the ultraviolet light LUV is constant, when the level of the lethal dose is higher, the irradiation time is longer. Therefore, the field disinfection mobile robot 100 moves more slowly, and the amount of time that the field disinfection mobile robot 100 stays in the space to be disinfected is longer. Conversely, when the level of the lethal dose is lower, the irradiation time is shorter. Therefore, the field disinfection mobile robot 100 moves faster, in other words, the amount of time that the field disinfection mobile robot 100 stays in the space to be disinfected is shorter.

In other embodiments, the processing circuit 123 may adjust the intensity of the ultraviolet light LUV emitted by the lamp 111. For example, when the irradiation time (for example, 5 minutes) calculated by the processing circuit 123 exceeds a maximum value (for example, 3 minutes), the processing circuit 123 sets the irradiation time to be equal to the maximum value and increases the intensity of the ultraviolet light LUV according to the level of the lethal dose. Similarly, when the irradiation time (for example, 30 seconds) is less than a minimum value (for example, as 1 minute), the processing circuit 123 sets the irradiation time to be equal to the minimum value, and decreases the intensity of the ultraviolet light LUV according to the level of the lethal dose. In some embodiments, the processing circuit 123 calculates appropriate irradiation time according to the level of the lethal dose and appropriately adjusts the intensity of the external light LUV.

In the embodiment, the processing circuit 123 divides the level of lethal dose by the intensity of the ultraviolet light LUV to obtain irradiation time. For example, in a case where the intensity of the ultraviolet light LUV is constant, when the level of the lethal dose is higher, the irradiation time is longer. Conversely, when the level of the lethal dose is lower, the irradiation time is shorter. In the embodiment, the processing circuit 123 controls the rotation speeds of the wheels 121 and 122 based on the calculated irradiation time. For example, when the irradiation time is longer, the rotation speed of the wheels 121 and 122 is less. When the irradiation time is shorter, the rotation speed of the wheels 121 and 122 is greater.

In an embodiment, the mobile device 120 further comprises a driving circuit (not shown) for controlling the rotation speed and turning direction of the wheels 121 and 122. In the embodiment, the processing circuit 123 may control the rotation speed and turning direction of the wheels 121 and 122 through the driving circuit. The invention does not intend to limit the number of wheels of the mobile device 120. In an embodiment, the mobile device 120 comprises more or fewer scroll wheels. In some embodiments, the wheels 121 and 122 are implemented by omni wheels, which can turn toward many different directions.

In an embodiment, the mobile device 120 comprises an input interface 124 for the user to input a level for the lethal dose. The invention does not intend to limit the type of the input interface 124. In the embodiment, the input interface 124 comprises buttons 125-127. The buttons 125˜127 correspond to different levels for the lethal dose. In the embodiment, the case where the button 125 is pressed indicates that the user wants to eliminate bacteria A in the air. Therefore, the processing circuit 123 searches a lookup table (LUT) to read a first level for the lethal dose and obtains first irradiation time by using the first level of the lethal dose. In this case, the processing circuit 123 commands the wheels 121 and 122 to rotate at a first rotation speed. The case where the button 126 is pressed indicates that the user wants to eliminate bacteria B in the air. Therefore, the processing circuit 123 searches the look-up table to read a second level for the lethal dose and obtains second irradiation time by using the second level of the lethal dose. In this case, the processing circuit 123 commands the wheels 121 and 122 to rotate at a second rotation speed. The case where the button 127 is pressed indicates that the user wants to eliminate bacteria A in the air. Therefore, the processing circuit 123 searches the look-up table to read a third level for the lethal dose and obtains third irradiation time by using the third level of the lethal dose. In this case, the processing circuit 123 indicates the wheels 121 and 122 to rotate at a third rotation speed.

In another embodiment, the input interface 124 is a numeric keyboard (not shown). The user inputs a level for the lethal dose by using the numeric keyboard. In the embodiment, the processing circuit 123 calculates the irradiation time based on the level of the lethal dose as input by the user. In some embodiments, the input interface 124 is a connection port (not shown), such as a USB port or a wireless receiver. In these embodiments, the user may input a level for the lethal dose in a wired or wireless manner.

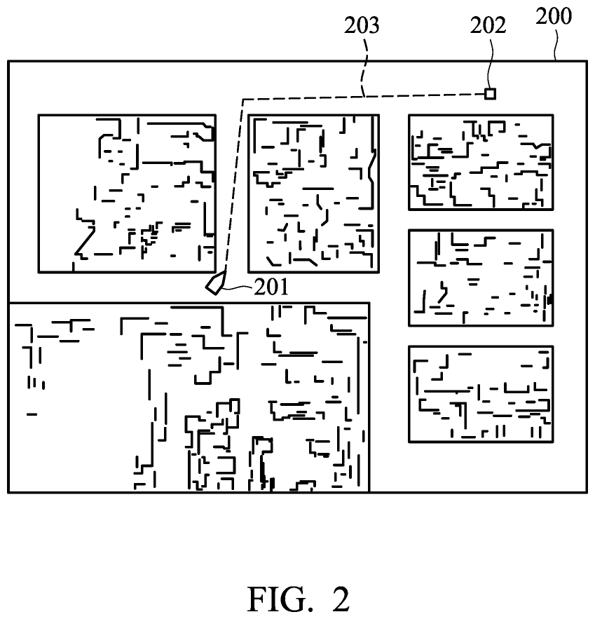

In other embodiments, the mobile device 120 further comprises a display panel 128 which shows a map. FIG. 2 is a schematic diagram of a map according to an embodiment of the present invention. The user may select a location on a map 200 as an end point 202 through the input interface 124. In an embodiment, the processing circuit 123 calculates the irradiation time and plans a walking path 203 based on the level of the lethal dose, the intensity of the ultraviolet light LUV, the location of the field disinfection mobile robot 100 (or start point 201), and the end point 202. In the embodiment, the field disinfection mobile robot 100 moves along the walking path 203 and stops at the end point 202. The invention does not intend to limit how the map is generated. In an embodiment, the user inputs the map 200 to the processing circuit 122 through the input interface 124. In another embodiment, the map 200 is generated by the processing circuit 122. In the embodiment, the processing circuit 122 may generate and update the map 200 in real time by using a Simultaneous Localization and Mapping (SLAM) technology.

Referring to FIG. 1, the mobile device 120 may further comprises a sensing circuit 129. The sensing circuit 129 detects the number of people in the space where the field disinfection mobile robot 100 is located and generates a value according to the detected result. In an embodiment, the sensing circuit 129 comprises at least one infrared sensor. In other embodiments, a counter (not shown) for counting the number of people provides a value which indicates the number of people to the processing circuit 123. In these embodiments, the counter for counting the number of people is independent of the field disinfection mobile robot 100. The processing circuit 123 may receive the counting result generated by the external counter for counting the number of people in a wireless or wired manner. In an embodiment, the user inputs a value which indicates the number of people to the processing circuit 123 through the input interface 124.

In the embodiment, the processing circuit 123 controls the operation mode of the light-emitting device 110 according to a value which indicates the number of people, so that the light-emitting device 110 operates in an air disinfection mode or a surface disinfection mode. In an embodiment, the sensing circuit 129 operates to detect a congestion level of a passageway or the number of people wearing masks. In this embodiment, the processing circuit 123 controls the operation mode of the light-emitting device 110 according to the detection result of the sensing circuit 129. In some embodiments, the processing circuit 123 further supplies power to the light-emitting device 110 to light the lamp 111 and controls the intensity of the ultraviolet light LUV of the lamp 111.

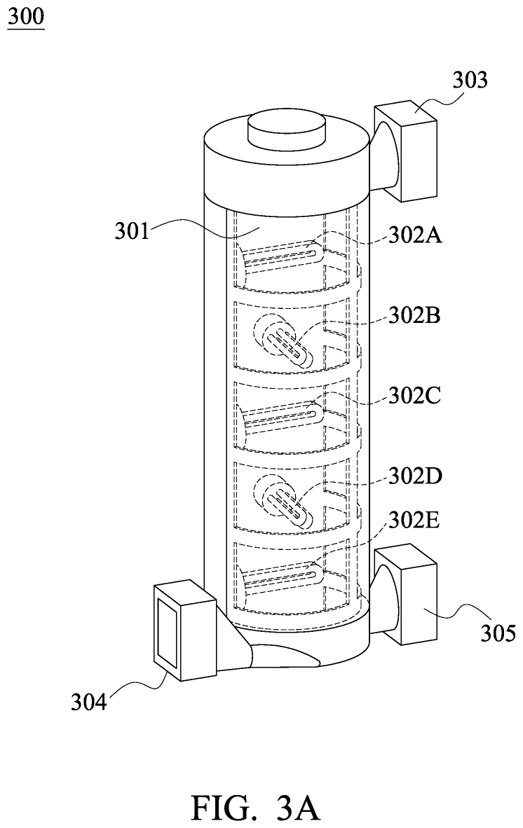

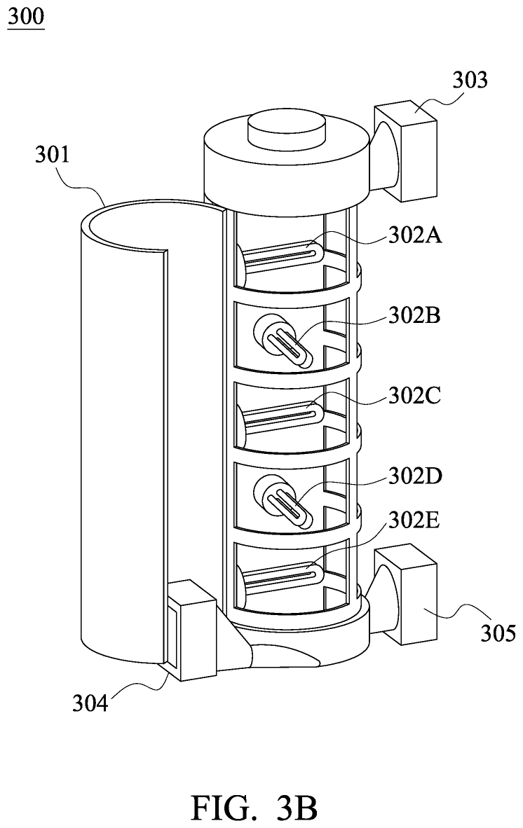

FIG. 3A is a schematic diagram of a state of a light-emitting device according to an exemplary embodiment of the present invention. In the embodiment, a cover 301 of a light-emitting device 300 operates in a closed state. As shown in FIG. 3A, the light-emitting device 300 comprises lamps 302A-302E which are disposed in the light-emitting device 300 and further comprises an air outlet 303 and air inlets 304 and 305 which are disposed outside of the light-emitting device 300. The cover 301 can be stretched, opened, and closed, so that the ultraviolet light emitted by the lamps 302A-302E is exposed or not exposed. The invention does not intend to limit the number of lamps of the light-emitting device 300. In other embodiments, the light emitting device 300 may comprise more or fewer lamps.

For example, when the value which indicates the number of people reaches a threshold, the processing circuit 123 commands the cover 301 to block the ultraviolet light emitted by the lamps 302A-302E. At this time, the light-emitting device 300 operates in the air disinfection mode. In this mode, the lamps 302A-302E are in a closed space, and the ultraviolet light emitted by the lamps 302A-302E are not transmitted out of the light emitting device 300. Therefore, the air entering the light-emitting device 300 flows through the lamps 302A-302E. Since the ultraviolet light emitted by the lamps 302A-302E provides a sterilization function, the air which has flowed through the lamps 302A-302E becomes clean air.

In an embodiment, the processing circuit 123 lights at least one lamp according to the level of the lethal dose and the intensity of the ultraviolet light emitted by each lamp. For example, when the level of the lethal dose is higher, more lamps are lit. When the level of the lethal dose is lower, fewer lamps are lit.

In other embodiments, the processing circuit 123 turns on a motor (not shown) to suck in air through the air inlets 304 and 305. Since the cover 301 covers the lamps 302A-302E, the air flows through the lamps 302A-302E and is expelled through at the air outlet 303. Since the ultraviolet light emitted by the lamps 302A-302E destroys bacteria or viruses in the air sucked in through the air inlets 304 and 305, the air expelled through the air outlet 303 is clean (aseptic) air.

The present invention does not intend to limit the number of air inlets of the light-emitting device 300. In other embodiments, the light-emitting device 300 may comprise more or fewer air inlets. In these examples, the positions of the air inlets are lower than the position of the air outlet, that is, the air inlets are closer to the mobile device than the air outlet. The invention also does not intend to limit the number of air outlets. In an embodiment, the light-emitting device 300 may comprise more air outlets.

FIG. 3B is a schematic diagram of another state of a light-emitting device according to an exemplary embodiment of the present invention. In the embodiment, the outer cover 301 operates in an open state. When the value which indicates the number of people does not reach a threshold, the processing circuit 123 commands the light-emitting device 300 to be opened the cover 301. Therefore, the ultraviolet light emitted by the lamps 302A-302E is transmitted out of the light-emitting device 300. At this time, the light emitting device 300 enters the surface disinfection mode. In this mode, since the ultraviolet light emitted by the lamps 302A-302E irradiates the surfaces of the objects around the field disinfection mobile robot 100, the bacteria or viruses on the surfaces of the objects can be disinfected.

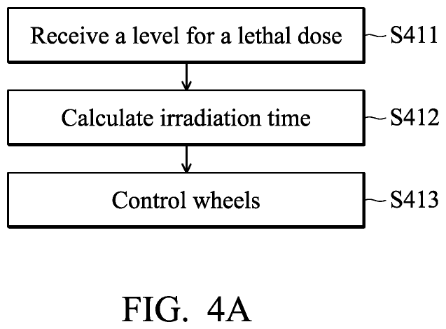

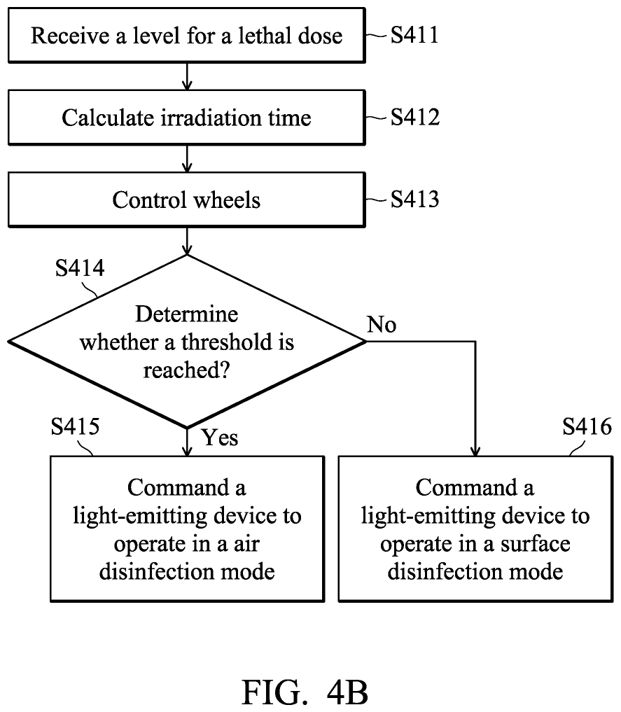

FIG. 4A is a schematic flow chart of a control method according to one exemplary embodiment of the present invention. The control method of the present invention can be applied to the field disinfection mobile robot 100 shown in FIG. 1. First, a level for a lethal dose is received (Step S411). In an embodiment, the field disinfection mobile robot 100 comprises an input interface 124 for receiving the level of the lethal dose. The input interface may comprise a plurality of buttons. Different buttons represent different levels for the lethal dose. In other embodiments, the input interface may be implemented by a numeric keyboard or a connection port.

According to the level of the lethal dose and the intensity of the ultraviolet light, irradiation time is calculated (Step S412). In an embodiment, the processing circuit 123 divides the level of the lethal dose by the intensity of the ultraviolet light LUV to obtain the irradiation time. For example, in a case where the intensity of ultraviolet light LUV is constant, when the level of the lethal dose is higher, the irradiation time is longer. Conversely, when the level of the lethal dose is lower, the irradiation time is shorter.

Next, the rotation speed of the wheels is controlled according to the irradiation time (Step S413). In an embodiment, the rotation speed of the wheels is inversely proportional to the irradiation time and also inversely proportional to the level of the lethal dose. For example, when the level of the lethal dose is higher, the rotation speed of the wheels is less due to the longer irradiation time. Therefore, the residence time of the field disinfection mobile robot 100 becomes longer. Conversely, when the level of the lethal dose is lower, the rotation speed of the wheels is greater due to the shorter irradiation time. Therefore, the residence time of the field disinfection mobile robot 100 becomes shorter.

In other embodiments, the intensity of the ultraviolet light LUV is controlled in Step S413. In these embodiments, when the irradiation time required by the processing circuit 123 is too long, the processing circuit 123 may increase the intensity of the ultraviolet light LUV to reduce the irradiation time. Similarly, when the irradiation time required by the processing circuit 123 is too short, the processing circuit 123 may decrease the intensity of the ultraviolet light LUV to increase the irradiation time.

In some embodiments, the turning direction of the wheels is also controlled in Step S413. For example, when the user marks the end point 202 in the map 200 shown on the display panel 128, the processing circuit 123 also considers the location of the field disinfection mobile robot 100 (that is, the start point 201) and the end point 202 for calculating the irradiation time. Moreover, the processing circuit 123 also plans the walking path 203 according to the level of the lethal dose, the intensity of the ultraviolet light LUV, the start point 201, and the end point 202 and controls the turning direction of the wheels 121 and 122 based on the walking path 203.

FIG. 4B is a schematic flow chart of a control method according to another exemplary embodiment of the present invention. The embodiment of FIG. 4B is similar to the embodiment of FIG. 4A, except additional steps S414˜S416 of FIG. 4B. In the embodiment, whether the value indicating the number of people is greater than a threshold is determined in Step S414. The present invention does not intend to limit the manner in which the value indicating the number of people is generated. In an embodiment, the value indicating the number of people is generated by the sensing circuit 129. The sensing circuit 129 operates to count the number of people around the field disinfection mobile robot 100. In the embodiment, the sensing circuit 129 is disposed in the field disinfection mobile robot 100. In another embodiment, the value indicating the number of people is provided by a counter (not shown) for counting the number of people. In the embodiment, the counter for counting the number of people is independent of the field disinfection mobile robot 100. According to other embodiments, in Step S414, whether a congestion level of a passageway or the number of people wearing masks is greater than a threshold.

When the value indicating the number of people reaches the threshold, the light-emitting device enters the air disinfection mode (Step S415). In the air disinfection mode, the processing circuit 123 commands the cover 301 of the light emitting device 300 to be closed. Therefore, the air which is sucked in through the air inlets 304 and 305 flows through the lamps 302A to 302E and is then expelled through the air outlet 303. Since the ultraviolet light emitted by the lamps 302A-302E provides a sterilization function, the air expelled at the air outlet 303 is clean air. In another embodiments, the processing circuit 123 lights at least one of the lamps 302A-302E according to the level of the lethal dose and the intensity of the ultraviolet light emitted by the lamps 302A-302E. In the embodiment, the more lamps are lit, the greater the intensity of ultraviolet light.

When the value indicating the number of people does not reach the threshold, the light-emitting device enters the surface disinfection mode (Step S416). In the surface disinfection mode, the processing circuit 123 controls the cover 301 of the light emitting device 300 to be opened. When the cover is opened, the ultraviolet light emitted by the lamps 302A to 302E irradiates the surfaces of the objects around the field cleaning mobile robot 100.

Control methods, or certain aspects or portions thereof, may take the form of a program code (i.e., executable instructions) embodied in tangible media, such as floppy diskettes, CD-ROMS, hard drives, or any other machine-readable storage medium, wherein, when the program code is loaded into and executed by a machine such as a computer, the machine thereby becomes processing circuits for practicing the methods. The methods may also be embodied in the form of a program code transmitted over some transmission medium, such as electrical wiring or cabling, through fiber optics, or via any other form of transmission, wherein, when the program code is received and loaded into and executed by a machine such as a computer, the machine becomes processing circuits for practicing the disclosed methods. When implemented on a general-purpose processor, the program code combines with the processor to provide a unique apparatus that operates analogously to application-specific logic circuits.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). For example, it should be understood that the system, device and method may be realized in software, hardware, firmware, or any combination thereof. Therefore, the scope of the appended claims should be accorded the broadest interpretation so as to encompass all such modifications and similar arrangements.

What is claimed is:

1. A field disinfection mobile robot comprising:
a light-emitting device comprising at least one lamp, wherein the at least one lamp is configured to emit ultraviolet light; and
a mobile device carrying the light-emitting device and comprising:
at least one wheel; and
a processing circuit calculating irradiation time according to a level of a lethal dose and intensity of the ultraviolet light and controlling a rotation speed of the at least one wheel according to the irradiation time.

2. The field disinfection mobile robot as claimed in claim 1, wherein the light-emitting device comprises:
a cover covering the at least one lamp;
at least one air inlet configured to suck in air; and
at least one air outlet configured to expel air,
wherein in response to the cover being closed, air sucked in through the at least one air inlet flows through the at least one lamp and is expelled through the at least one air outlet, and
wherein in response to the cover being opened, the ultraviolet light irradiates surfaces of objects around the field disinfection mobile robot.

3. The field disinfection mobile robot as claimed in claim 2,
wherein in response to a value which indicates the number of people reaching a threshold, the processing circuit commands the cover to be closed, and
wherein in response to the value which indicates the number of people not reaching the threshold, the processing circuit commands the cover to be opened.

4. The field disinfection mobile robot as claimed in claim 3, wherein the mobile device further comprises:
a sensing circuit configured to detect the number of people around the field disinfection mobile robot and generate the value which indicates the number of people.

5. The field disinfection mobile robot as claimed in claim 3, wherein the value which indicates the number of people is provided by a counter, and the counter is independent of the field disinfection mobile robot.

6. The field disinfection mobile robot as claimed in claim 1, wherein the mobile device further comprises:
an input interface, wherein a user inputs the level of the lethal dose through the input interface.

7. The field disinfection mobile robot as claimed in claim 6, wherein the input interface comprises a plurality of buttons which respectively correspond to different levels for the lethal dose.

8. The field disinfection mobile robot as claimed in claim 6, wherein the mobile device further comprises:
a display panel showing a map,
wherein the user selects a location on the map as an end point of the field disinfection mobile robot through the input interface, and
wherein the processing circuit calculates the irradiation time according to the level of the lethal dose, the intensity of the ultraviolet light, and a start point and the end point of the field disinfection mobile robot.

9. The field disinfection mobile robot as claimed in claim 1, wherein in response to the light-emitting device comprising a plurality of lamps, the processing circuit lights at least one of the plurality of lamps according to the level of the lethal dose and the intensity of the ultraviolet light emitted by each lamp.

10. The field disinfection mobile robot as claimed in claim 1, wherein the level of the lethal dose is inversely proportional to the rotation speed of the at least one wheel.

11. A control method for a field disinfection mobile robot, the field disinfection mobile robot comprising a light-emitting device and at least one wheel, the light-emitting device comprising at least one lamp and a cover, the cover covering the at least one lamp, the at least one lamp configured to emit ultraviolet light, and the control method comprising:
receiving a level for a lethal dose;
calculating irradiation time according to the level of the lethal dose and intensity of the ultraviolet light; and
controlling a rotation speed of the at least one wheel according to the irradiation time.

12. The control method as claimed in claim 11, wherein the level of the lethal dose is inversely proportional to the rotation speed of the at least one wheel.

13. The control method as claimed in claim 11,
wherein in response to the cover being closed, air which is sucked in through an air inlet flows through the at least one lamp and is expelled through an air outlet, and
wherein in response to the cover being opened, the ultraviolet light irradiates surfaces of objects around the field disinfection mobile robot.

14. The control method as claimed in claim 12, further comprising:
controlling the cover according to a value which indicates the number of people;
in response to the value which indicates the number of people reaching a threshold, closing the cover, and
in response to the value which indicates the number of people not reaching the threshold, opening the cover.

15. The control method as claimed in claim 14, further comprising:
enabling a sensing circuit to detect the number of people around the field disinfection mobile robot and generate the value which indicates the number of people,
wherein the sensing circuit is disposed in the field disinfection mobile robot.

16. The control method as claimed in claim 14,
wherein the value which indicates the number of people is provided by a counter, and
wherein the counter is independent of the field disinfection mobile robot.

17. The control method as claimed in claim 11, wherein a level for the lethal dose is received through an input interface of the field disinfection mobile robot, wherein a user inputs the level for the lethal dose through the input interface.

18. The control method as claimed in claim 17, wherein the input interface comprises a plurality of buttons which respectively correspond to different levels for the lethal dose.

19. The control method as claimed in claim 17, further comprising:
showing a map,
wherein the user selects a location on the map as an end point of the field disinfection mobile robot through the input interface, and
wherein the irradiation time is calculated according to the level of the lethal dose, the intensity of the ultraviolet light, and a start point and the end point of the field disinfection mobile robot.

20. The control method as claimed in claim 11, wherein in response to the light-emitting device comprising a plurality of lamps, at least one of the plurality of lamps is lit according to the level of the lethal dose and the intensity of ultraviolet light emitted by each lamp.
